(19) United States
(12) Patent Application Publication
(10) Pub. No.: US 2022/0057416 A1
(43) Pub. Date: Feb. 24, 2022

(54) METHOD FOR OPTIMIZATION OF CANNABIS DOSAGE AND MIXTURE OF ACTIVE CANNABINOIDS

(71) Applicant: THSEE, LLC, Palm Beach Gardens, FL (US)

(72) Inventor: Alan DIAMOND, Palm Beach Gardens, FL (US)

(21) Appl. No.: 17/417,070

(22) PCT Filed: Dec. 23, 2019
(86) PCT No.: PCT/US2019/068420

§ 371 Date: Jun. 21, 2021

Related U.S. Application Data
(60) Provisional application No. 62/784,058, filed on Dec. 21, 2018.

(51) Int. Cl. G01N 33/94; G16H 20/10; G16H 10/40

(52) U.S. Cl. CPC G01N 33/948; G16H 10/40; G16H 20/10

(57) ABSTRACT

A home testing kit used to collect a blood sample of the user and a method of blood analysis used to calculate correct mixtures of THC and CBD for each patient's desired therapeutic benefit.

### FIELD OF THE INVENTION

The invention relates to a method for optimization of cannabis usage to achieve desired therapeutic benefits while minimizing undesirable side effects. This method is based upon measuring levels of cannabidiol and tetrahydrocannabinol, along with other biomarkers, for metabolism in a patient's blood sample and analyzing this data to alter a user or a medical patient's cannabis dosage with relation to relative concentrations of cannabidiol and tetrahydrocannabinol. The invention further relates to a kit suitable for home collection of a user's or patient's blood sample.

### BACKGROUND OF THE INVENTION

Cannabis has long been known to have medicinal properties across a variety of modalities, including pain relief, treatment of anxiety and depression, improved sleep, increased focus, as an antiemetic, an antispasmodic, and for the treatment of epileptic seizures. These properties are derived from the presence of chemicals naturally found in the plant Cannabis satvia (Indian hemp): cannabidiol (CBD), tetrahydrocannabinol (THC), and other phytocannabinoids (El Sohly M A and Slade D, (2005) Life Sci, 78:539-548 and Huestis M A, (2005) Handb Exp Pharmacol, 168:657-690). Current medical research is only now beginning to appreciate the beneficial properties of these compounds which have previously only been characterized by anecdotal reports. Specifically, current research has focused on the therapeutic targets of cannabinoids with CBD and THC showing the most promise as potential therapeutic compounds (Ibeas Bih C, (2015) Neurotherapeutics, 12(4):699-730).

An important step in designing efficacious therapies for cannabinoids is understanding the pharmacokinetics of CBD and THC, both as monotherapy, and in combination. Each of these compounds has their own molecular targets within the body (Grotenhermen F, (2003) Clin Pharmacokinet, 42(4) 327-260). Varying the ratio of THC and CBD produces different benefits for the patient. For example, a 1:1 ratio of THC to CBD is most commonly recommended for relief of pain and nausea, whereas for the treatment of seizures and inflammation, a 1:2 ratio, is preferable.

Variations in the metabolisms of individual patients may also affect how rapidly cannabinoids are processed in the body. This presents a challenge in the design of efficacious cannabinoid therapies, specifically, what mixtures of THC and CBD are required for intake to achieve the desired concentrations in vivo to achieve the therapeutic goal.

There is need in the art for a rapid means of testing for biomarkers of cannabinoid metabolism coupled with a method to use this information to calculate correct mixtures of THC and CBD for each patient's desired therapeutic benefit.

### BRIEF SUMMARY OF THE INVENTION

The invention is considered to be a method for the optimization of a dosage of cannabis, based upon the levels of THC and CBD in a user's or patient's blood sample, along with the levels of certain biomarkers and metabolites relevant to the metabolism of THC and CBD.

The invention further comprises a kit for the drawing of a blood sample at home which may then be shipped to an offsite lab to perform the necessary testing. In various embodiments, the kit comprises any or all of: a safety lancet, microfilter paper, antiseptics, an alcohol swab, a suitable wound dressing following blood draw, a suitable storage container for the blood sample, and a pre-stamped, pre-addressed envelope for submission of sample for testing.

The invention improves over the prior art by making a better recommendation as to which strains of cannabis may provide greater therapeutic benefit with fewer side effects based upon the patient's metabolism.

### DETAILED DESCRIPTION OF THE INVENTION

### Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The articles “a” and “an” are used herein to refer to one or to more than one (i.e., to at least one) of the grammatical object of the article. The term “and/or” as used herein is defined as the possibility of having one or the other or both. For example, “A and/or B” provides for the scenarios of having just A or just B or a combination of A and B. If the claim reads A and/or B and/or C, the composition may include A alone, B alone, C alone, A and B but not C, B and C but not A, A and C but not B or all three A, B and C as components.

As used herein, the term “active form” refers to the metabolite form of the inactive prodrug that is metabolized within the body into its active form.

As used herein the terms “ameliorate,” “ameliorated,” and “amelioration,” which may be used interchangeably and as used herein, refers to the ability to make better or more tolerable.

As used herein the term “biomarker” refers to a measurable substance in an organism whose presence is indicative of some phenomenon such as disease, infection or environmental exposure.

As used herein, the term “cannabinoids” refers to terpeno-phenolic compounds.

As used herein, the terms “endogenous cannabinoids and “endocannabinoids” are used interchangeably, and refer to any substances produced from within the body that activate cannabinoid receptors, including, but not limited to: anandamide, arachidonoylethanolamine, 2-arachidonoylglycerol (2-AG), 2-arachidonyl glyceryl ether, N-nrachidonoyl dopamine, virodhamine, lysophosphatidylinositol, and metabolites thereof.

As used herein, the term “exogenous cannabinoid” refers to any substances not produced by the body that activate cannabinoid receptors, including, but not limited to: tetrahydrocannabinol (THC), cannabidiol (CBD), cannabinol (CBN), cannabigerol (CBG), cannabidiolic acid (CBDA), tetrahydrocannabivarin (THCV), cannabidivarin (CBDV), cannabichromene (CBC), 11-Hydroxy-Δ9-tetrahydrocannabinol (11-OH-THC), 11-Nor-9-carboxy-Δ9-tetrahydrocannabinol (11-COOH-THC or THC-COOH), tetrahydrocannabinolic acid (THCA), hydroxylated 7-COOH derivatives of cannabadiol, hydroxylated 7-OH derivatives of cannabadiol, aminoalkylindoles, 1,5-diarylpyrazoles, quinolines, arylsulfonamides, and metabolites thereof.

As used herein, the term “cannabis usage” refers to the action, amount or mode of ingestion of cannabis by the patient.

As used herein, the terms “ingest,” “ingested,” “ingesting,” and “ingestion,” which may be used interchangeably and as used herein, refer to the act of intaking cannabis into the body by any available means, such as by oral intake with natural digestion, inhalation, transdermal absorption and the like.

As used herein, the terms “inhaled administration,” “inhale,” “inhaled,” “inhalation,” and “inhalation therapy,” which may be used interchangeably and as used herein, include administration of a substantially uniform distribution of appropriately sized particles to the respiratory epithelium of the nose, central airways, the peripheral aspect of the lung and/or the alveolar region of the lung or by intratracheal instillation. Common forms of delivery via inhalation include burning the cannabis and breathing in the resulting smoke or by heating a liquid form of cannabis (such as an oil) to generate an aerosol, commonly called a “vapor”, that the user inhales.

As used herein, the term “optimal” refers to the best or most favorable option of several available options.

As used herein, the term “personalized” refers to the act of making or altering so as to meet individual needs, inclinations or specifications.

As used herein, the terms “predetermine” and “predetermined,” which may be used interchangeably and as used herein, refer to something established or decided in advance.

As used herein, the term “subjective parameters” refers to numerical or other measurable factors forming one of a set that defines a system or sets the conditions of operation that are peculiar to a particular individual.

As used herein, the term the terms “suppress” and “suppression,” which may be used interchangeably and as used herein, refer to the act of preventing the development, action or expression of an undesirable condition in a patient.

As used herein, the term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions of the present invention may be administered in a sufficient amount to produce a at a reasonable benefit/risk ratio applicable to such treatment.

As used herein, the term “trained algorithm” refers to an algorithm developed using a reference set of known cannabis-related biomarkers.

As used herein, the term “variant” refers to a form or version of something that differs in some respect from other forms of the same thing or from a standard.

Within the framework of the present description and in the subsequent claims, except where otherwise indicated, all numbers expressing amounts, quantities, percentages, and so forth, are to be understood as being preceded in all instances by the term “about.” As used herein, the term “about” is defined as ±5%. Also, all ranges of numerical entities include all the possible combinations of the maximum and minimum numerical values and all the possible intermediate ranges therein, in addition to those specifically indicated hereafter.

The invention is drawn to a method for improving the outcome of cannabis therapy by creating a personalized profile and recommending the optimal strains and/or types of cannabis to use based upon a patient's blood sample to achieve a patient's predetermined goals. The invention also encompasses a kit suitable for taking a fluid sample at home and shipment of the fluid sample to an offsite testing laboratory. To achieve this result, the inventor has developed a proprietary algorithm for a programmed computer that analyzes a patient's blood sample, including measuring tetrahydrocannabinol and cannabidiol and variants thereof levels, identifying certain cannabis-related biomarker levels, recognizing various subjective parameters of the patient and utilizing a detailed database of metabolic biomarkers and drug metabolites.

Efficacious cannabis therapy requires that specific levels of desired cannabinoids, typically THC, CBD, and their metabolites, remain in the body of the patient to achieve the desired predetermined goals and therapeutic outcome for a patient in need of treatment that cannabis is known to treat. Upon consumption of cannabis, THC and CBD are metabolized most commonly into 11-hydroxytetrahydrocannabinol (11-OH-THC), 11-nor-9-carboxy-tetrahydrocannabinol (THC-COOH) (Huestis M A, (2007) Chem Biodivers, 4(8): 1770-1804) and hydroxylated 7-COOH derivatives of CBD (Ujvary I and Hanus L, (2016) Cannabis Cannabinoid Res, 1(1)90-101). The pharmacokinetics of both CBD and THC are complex, and based upon numerous factors unique to each patient, most commonly cytochrome P450 enzymes in the liver (Jiang R et al., (2011) Life Sci, 89(5-6):165-70). Variations in individual metabolisms can cause high amount of variance in how both CBD and THC are processed within the body, as well as their ability to bind various molecular receptors (Thomas B F, (2017) Subst Abuse, 11:1-9 and Xiong W et al., (2012) J Exp Med, 209(6):1121-34). These factors cause a high degree of unpredictability when prescribing cannabis as therapy. The prior art is silent as to how the metabolism of CBD and THC can be used to improve the therapeutic benefits of cannabis therapy.

To overcome this problem, the inventors have created a list of metabolic biomarkers and drug metabolites found in a blood sample of a user who has recently consumed cannabis. By taking a blood sample from such a user, the levels of each metabolite can be computed by standard analytical methods, with particular emphasis on the ratio of THC to CBD, to create a personalized report that can recommend which strains of cannabis may prove more beneficial in achieving the user's goals, based upon the relative amounts of THC and CBD in the prescribed cannabis strain. In an embodiment, the user is a patient in need of treatment by optimization of cannabis strains to meet their therapeutic goals.

A personalized recommendation for each user is created by the analysis of various biomarkers present in a blood sample, taken after recent consumption of cannabis. In an embodiment, the cannabinoid related biomarkers are drawn from the group consisting of anandamide, arachidonoylethanolamine, 2-arachidonoylglycerol (2-AG), 2-arachidonyl glyceryl ether, N-nrachidonoyl dopamine, virodhamine, lysophosphatidylinositol, tetrahydrocannabinol (THC), cannabidiol (CBD), cannabinol (CBN), cannabigerol (CBG), tetrahydrocannabivarin (THCV), cannabidiolic acid (CBDA), cannabidivarin (CBDV), cannabichromene (CBC), 11-Hydroxy-Δ9-tetrahydrocannabinol (11-OH-THC), 11-Nor-9-carboxy-Δ9-tetrahydrocannabinol (11-COOH-THC or THC-COOH), tetrahydrocannabinolic acid (THCA), hydroxylated 7-COOH derivatives of cannabadiol, hydroxylated 7-OH derivatives of cannabadiol, aminoalkylindoles, 1,5-diarylpyrazoles, quinolines, arylsulfonamides, and metabolites thereof, the ratio of THC:CBD, high-density lipoprotein (HDL), low-density lipoprotein (LDL), triglycerides, high-sensitivity C-reactive protein (Hs-CRP), testosterone, glucose, hemoglobin A1c (HbA1C), thyroid stimulating hormone (TSH), cortisol, prolactin, omega-3 fatty acids, and omega-6 fatty acids.

The endocannabinoid system is a biological system composed of endocannabinoids which are endogenous lipid-based retrograde neurotransmitters that bond to cannabinoid receptors and cannabinoid receptor proteins that are expressed throughout the vertebrate central nervous system and peripheral nervous system. More particularly, the endocannabinoid system (ECS) is a widespread neuromodulatory system that plays important roles in central nervous system (CNS) development, synaptic plasticity, and the response to endogenous and environmental insults. The ECS is comprised of cannabinoid receptors, endogenous cannabinoids (endocannabinoids) and the enzymes responsible for the synthesis and degradation of the endocannabinoids. The most abundant cannabinoid receptor is the CB1 cannabinoid receptors, however CB2 cannabinoid receptors, transient receptor potential (TRP) channels, and peroxisome proliferator activated receptors (PPAR's) are also engaged by some cannabinoids. Exogenous cannabinoids, such as tetrahydrocannabinol, produce their biological effects through their interactions with cannabinoid receptors.

The inventor of the instant invention tested 2-arachidonoyl glycerol (2-AG) and arachidonoyl ethanolamide (anandamide), two recognized endogenous cannabinoids. Despite similarities in chemical structure, 2-AG and anandamide are synthesized and degraded by distinct enzymatic pathways, which impart fundamentally different physiological and pathophysiological roles to these two endocannabinoids.

2-Arachidonoylglycerol (2-AG) is an endocannabinoid, an endogenous agonist of the CB₁ receptor and the primary endogenous ligand for the CB2 receptor. It is an ester formed from the omega-6 fatty acid arachidonic acid and glycerol. It is present at relatively high levels in the central nervous system, with cannabinoid neuromodulatory effects. The activities of phospholipase C (PLC) and diacylglycerol lipase (DAGL) mediate its formation. 2-AG is synthesized from arachidonic acid-containing diacylglycerol (DAG).

Anandamide, also known as N-arachidonoylethanolamine (AEA), is a fatty acid neurotransmitter derived from the non-oxidative metabolism of eicosatetraenoic acid (arachidonic acid), an essential omega-6 fatty acid. It is synthesized from N-arachidonoyl phosphatidylethanolamine by multiple pathways. It is degraded primarily by the fatty acid amide hydrolase (FAAH) enzyme, which converts anandamide into ethanolamine and arachidonic acid. Anandamide's effects can occur in either the central or peripheral nervous system. These distinct effects are mediated primarily by CB₁ cannabinoid receptors in the central nervous system, and CB₂ cannabinoid receptors in the periphery. The latter are mainly involved in functions of the immune system. Cannabinoid receptors were originally discovered as being sensitive to Δ⁹-tetrahydrocannabinol (Δ⁹-THC, commonly called THC), which is the primary psychoactive cannabinoid found in cannabis. The discovery of anandamide came from research into CB₁ and CB₂, as it was inevitable that a naturally occurring (endogenous) chemical would be found to affect these receptors. Anandamide has been shown to impair working memory in rats. Studies are under way to explore what role anandamide plays in human behavior, such as eating and sleep patterns, and pain relief.

The measurement of 2-AG and Anandamide can determine if a user or patient has optimal levels of either endogenous cannabinoids in their system, of if said used and/or patient has below “optimal” levels and/or is deficient. In an embodiment, the cannabinoid related biomarkers tested for are exogenous cannabinoids. In another embodiment, the cannabinoid related biomarkers tested for are endogenous cannabinoids.

Testing may also include information regarding the user's current medical state and their goals in cannabis therapy. In various embodiments, these factors may include the user's height, weight, body-mass index, age, current rate of cannabis consumption (once a day, once a week, once a month), strains of cannabis used, the method of ingestion of cannabinoid, the time elapsed since last ingestion of cannabinoid, desired therapeutic benefits, and side effects to avoid. In an embodiment, the desired therapeutic benefits may include pain relief, anxiety suppression, amelioration of depression, improved sleep, improved alertness and focus, gastrointestinal disorder relief and prevention of seizures. In an embodiment, the side effects to avoid may include paranoia, sleepiness, anxiety, dry mouth and bloodshot eyes.

The analysis of the above data is then compared to other known strains and types of cannabis, based upon the user's desired outcomes. Most typically, therapeutic benefit is usually found at a balanced ratio of THC to CBD (1:1), however, certain desired benefits may be better achieved with other ratios. Specifically:
High THC (2:1) ratio produces improved sleep and restores appetite.
High CBD (1:2) ratio produces relief from seizures and inflammation.
A balanced ratio produces relief from pain, anxiety, and depression.

The ratio of the symptom goal and the current strain of cannabis being consumed by the patient is compared to the ratio of THC to CBD present in the user's blood sample. If the blood test ratio is lower than the desired ratio, a strain with higher THC to CBD content is recommended. If the blood test ratio is higher than the desired ratio, a strain with lower THC to CBD content is recommended.

For example, a user with the goal of improved sleep by cannabis therapy is recommended a THC:CBD ratio of 2:1. The user's blood test shows a ratio of 1.5:1. The personalized report therefore suggests that the user increase the TCH:CBD ratio of the strain of cannabis they are using by about 33% (from 1.5:1 to 2:1). An appropriate strain can then be recommended.

In various embodiments, the cannabis strains that are recommended include indica, sativa, and hybrids. In further embodiments, the recommended strain includes additional factors such as individual cannabinoid presence within the strain, THC, THCA, THCV, CBD, CBG, CBN, CBC, individual terpene presence within the strain, a-Bisabolol, a-Pinene, b-Caryophyllene, Myrcene, Limonene, Linalool, Humulene, a-Terpineol and Eucalyptol.

Plasma contains an abundance of proteins many of which can be used as biomarkers, indicating the presence of certain diseases in an individual. Currently, 2D Electrophoresis is the primary method for discovery and detection of biomarkers in plasma. This involves the separation of plasma proteins on a gel by exploiting differences in their size and pl. Plasma samples must undergo preparation procedures for accurate results to be obtained using 2D Electrophoresis. These preparation procedures aim to remove contaminants that may interfere with detection of biomarkers, solubilize the proteins so they are able to undergo 2D Electrophoresis analysis, and prepare plasma with minimal loss of low concentration proteins, but optimal removal of high abundance proteins.

Blood fractionation is the process of fractionating whole blood, or separating it into its component parts. This is typically done by centrifuging the blood. The resulting components are: a clear solution of blood plasma in the upper phase, the buffy coat, which is a thin layer of leukocytes (white blood cells) mixed with platelets in the middle, and erythrocytes (red blood cells) at the bottom of the centrifuge tube. Serum separation tubes (SSTs) are tubes used in phlebotomy containing a silicone gel; when centrifuged the silicone gel forms a layer on top of the buffy coat, allowing the blood serum to be removed more effectively for testing and related purposes.

Plasma proteins are separated by using the inherent differences of each protein. Fractionation involves changing the conditions of the pooled plasma (e.g., the temperature or the acidity) so that proteins that are normally dissolved in the plasma fluid become insoluble, forming large clumps, called precipitate. The insoluble protein can be collected by centrifugation. One of the very effective ways for carrying out this process is the addition of alcohol to the plasma membrane pool while simultaneously cooling the pool. This process is sometimes called cold alcohol fractionation or ethanol fractionation.

A number of commercially available plasma serum separation devices may be used to process a blood sample obtained from a using a device for separating the serum included in a certain volume of blood by way of centrifugation (Vacunator®, Quest Diagnostics, Secaucus, N.J.).

### Kits

This disclosure also provides kits for conveniently and effectively implementing the methods disclosed herein. Such kits comprise any subject composition, and a means for facilitating compliance with methods disclosed herein. Such kits provide a convenient and effective means for the subject to collect a fluid sample, store said sample so that it is a viable candidate for analysis and means in which to successfully transport said sample to a designated laboratory for processing and analysis. The compliance means of such kits includes any means which facilitates the acquisition, storage and transport of a viable fluid sample that can be effectively analyzed according to a method disclosed herein. Such compliance means include instructions, packaging, and dispensing means, and combinations thereof. Kit components may be packaged for either manual or partially or wholly automated practice of the foregoing methods. In an embodiment, the fluid sample is saliva. In another embodiment, the fluid sample is blood. In other embodiments involving kits, the disclosure contemplates a kit including compositions disclosed herein, and optionally instructions for their use. One embodiment of a kit of the present invention, includes, but is not limited to, the following articles: a 17-21 gauge safety lancet, oral fluid collection swab, funnel, tube or syringe, binder-free microfiber paper, preferably cut by a laser cutter into ¼ wide by 1 inch long strips, one or more alcohol swabs, adhesive bandages, a biohazard sample storage bag, a pre-stamped, pre-addressed envelope to send a fluid sample to the testing laboratory and instructions on the use of the kit.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A method for processing and analyzing a blood or oral fluid sample to determine optimal cannabis usage to achieve predetermined goals of a user comprising the steps of:
in a programmed computer, inputting subjective parameters about a cannabis user and one or more predetermined goals of the user;
obtaining a fluid sample from said user;
subjecting said fluid sample to testing to indicate presence of cannabinoid related biomarkers in said sample;
measuring tetrahydrocannabinol and cannabidiol levels in said fluid sample;
identifying tetrahydrocannabinol and cannabidiol variants in said fluid sample;
in said programmed computer, inputting the names of identified cannabinoid related biomarkers, said tetrahydrocannabinol and cannabidiol levels and the names of any tetrahydrocannabinol and cannabidiol variants found in said blood sample;
to a trained algorithm, generating a profile of said fluid sample as to whether or not said patient is achieving said predetermined goals;
electronically outputting a personalized report that identifies which specific strains and/or types of cannabis can assist said user to achieve their predetermined goals, and
prescribing an optimal strain of cannabis to achieve said goals.

2. The method according to claim 1, wherein said cannabinoid related biomarkers are selected the group consisting of anandamide, arachidonoylethanolamine, 2-arachidonoylglycerol (2-AG), 2-arachidonyl glyceryl ether, N-nrachidonoyl dopamine, virodhamine, lysophosphatidylinositol, tetrahydrocannabinol (THC), cannabidiol (CBD), cannabinol (CBN), cannabigerol (CBG), tetrahydrocannabivarin (THCV), cannabidiolic acid (CBDA), cannabidivarin (CBDV), cannabichromene (CBC), 11-Hydroxy-09-tetrahydrocannabinol (11-OH-THC), 11-Nor-9-carboxy-A9-tetrahydrocannabinol (11-COOH-THC or THC-COOH), tetrahydrocannabinolic acid (THCA), hydroxylated 7-COOH derivatives of cannabadiol, hydroxylated 7-OH derivatives of cannabadiol, aminoalkylindoles, 1,5-diarylpyrazoles, quinolines, arylsulfonamides, and metabolites thereof, the ratio of THC:CBD, high-density lipoprotein (HDL), low-density lipoprotein (LDL), triglycerides, high-sensitivity C-reactive protein (Hs-CRP), testosterone, glucose, hemoglobin A1c (HbA1C), thyroid stimulating hormone (TSH), cortisol, prolactin, omega-3 fatty acids, and omega-6 fatty acids.

3. The method according to claim 1, wherein said cannabinoid related biomarkers are exogenous or endogenous cannabinoids.

4. (canceled)

5. The method according to claim 1, wherein said subjective parameters of the cannabis user are selected from the group consisting of age, height, weight, amount and types and/or strains of cannabinoid ingested by the cannabis user over a period of time selected from the group consisting of one day, one week, one month and one hour, the method of ingestion of cannabinoid, the time elapsed since last ingestion of cannabinoid, and treatment goals and side effects selected from the group consisting of paranoia, sleepiness, anxiety, dry mouth and bloodshot eyes, experienced by the cannabis user.

6. The method according to claim 1, wherein said predetermined goals of the user are selected from the group consisting of pain relief, anxiety suppression, amelioration of depression, improved sleep, improved alertness and focus, gastrointestinal disorder relief and prevention of seizures.

7. (canceled)

8. (canceled)

9. The method according to claim 1, wherein said strain of cannabis is selected from the group consisting of indica, sativa and hybrid, individual cannabinoid presence within the strain, THC, THCA, THCV, CBD, CBG, CBN, CBC, individual terpene presence within the strain, a-Bisabolol, a-Pinene, b-Caryophyllene, Myrcene, Limonene, Linalool, Humulene, a-Terpineol and Eucalyptol.

10. The method according to claim 1, wherein said programmed computer uses said algorithm to determine the ratio of tetrahydrocannabinol to cannabidiol in said blood sample provided by the user and to compare the calculated ratio to the identified tetrahydrocannabinol and cannabidiol variants in said blood sample, identified cannabinoid related biomarkers, endocannabinoids, exogenous cannabinoids, and any tetrahydrocannabinol and cannabidiol variants to determine the strain of cannabis to best achieve the user's predetermined goal.

11. (canceled)

12. The method according to claim 1, wherein said user is a patient in need of medical treatment.

13. A kit comprising:
one or more means in which to extract a fluid sample, wherein said fluid is saliva or blood; and
one or more means in which to store said fluid sample.

14. (canceled)

15. (canceled)

16. The kit according to claim 13, wherein said fluid extraction means is a safety lancet, swab, saliva collection tube or syringe.

17. The kit according to claim 16, wherein said safety lancet is a 17 to 21 gauge safety lancet.

18. The kit according to claim 13, wherein said one or more means in which to store said blood sample is a binder-free glass microfiber filter paper cut into strips that are ¼ inch wide by 1 inch long.

19. (canceled)

20. (canceled)

21. (canceled)

22. The kit according to claim 13, further comprising one or more antiseptics.

23. The kit according to claim 22, wherein said antiseptic is an alcohol swab.

24. The kit according to claim 13, further comprising one or more wound dressings.

25. The kit according to claim 24, wherein said wound dressing is an adhesive bandage.

26. (canceled)

27. (canceled)

28. The kit according to claim 13, further comprising instructions on how to take, store and handle said fluid sample.
